(19) United States
(12) Patent Application Publication
(10) Pub. No.: US 2007/0207189 A1
(43) Pub. Date: Sep. 6, 2007

(54) Antimicrobial medical devices

(76) Inventors: Nadya Belcheva, Middletown, CT (US); John Kennedy, Gullford, CT (US)

Correspondence Address: UNITED STATES SURGICAL,;A DIVISION OF TYCO HEALTHCARE GROUP LP, 195 MCDERMOTT ROAD, NORTH HAVEN, CT, 06473, US

(21) Appl. No.: 11/712,332

(22) Filed: Feb. 28, 2007

Related U.S. Application Data
(60) Provisional application No. 60/777,307, filed on Feb. 28, 2006.

(51) Int. Cl. A61K 9/70

(52) U.S. Cl. 424/443; 424/445

(57) ABSTRACT

Antimicrobial medical devices are prepared with a complexed antimicrobial agent which enhances the adherence of the antimicrobial agent to the medical device.

### CROSS-REFERENCE TO RELATED APPLICATIONS

This application claims the benefit of and priority to U.S. Provisional Patent Application No. 60/777,307 filed Feb. 28, 2006, the entire disclosure of which is incorporated by reference herein.

### TECHNICAL FIELD

The present disclosure relates to antimicrobial medical devices, and to methods for preparing and using such medical devices.

### DESCRIPTION OF RELATED ART

The use of antimicrobial agents on medical devices such as sutures and/or packages containing said sutures has been previously disclosed. However, some medical devices may not provide effective levels of antimicrobial activity for a sufficient period of time. Moreover, as is apparent from U.S. Patent Publication Nos. 2004/0068293 and 2004/0068294, antimicrobial agents on medical devices can be undesirably transferred to their packages, requiring the use of higher levels of antimicrobial agents in order to obtain the desired antimicrobial effect upon implantation of the suture or other medical device in vivo.

Accordingly, there is a need for medical devices that can remain in vivo for extended periods of time with enhanced antimicrobial efficacy. There is also a need for an easy and inexpensive method of applying an antimicrobial agent to a medical device that provides protection against microorganisms for extended periods of time with minimal loss of the antimicrobial agent from the device surface and/or minimal transference of the antimicrobial agent to packaging materials, etc., thus permitting the use of lower amounts of antimicrobial agents to achieve the desired antimicrobial effect in vivo.

### SUMMARY

Antimicrobial medical devices in accordance with this disclosure include a complexed antimicrobial agent located on at least a portion of a surface of the medical device. The complexed antimicrobial agent is provided on the medical device by applying an antimicrobial solution containing at least an antimicrobial agent, an adherence-enhancing agent, and a solvent. The complexed antimicrobial agent can be applied before, after, or simultaneously with a coating composition. In embodiments, the medical device may be a suture. In other embodiments, the present disclosure relates to methods wherein a suture having a complexed antimicrobial agent on at least a portion thereof is used to secure tissue or close a wound.

The complexed antimicrobial agent, by virtue of the adherence-enhancing agent, possesses greater affinity for the medical device to which it is applied, thereby reducing the loss of the antimicrobial agent from the surface of the medical device during handling, processing or storage, and thus providing for improved antimicrobial activity of the medical device upon implantation.

### BRIEF DESCRIPTION OF THE DRAWINGS

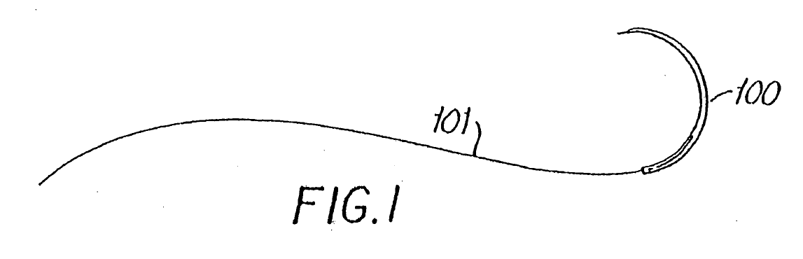

The Figure (depicted as FIG. 1) is a perspective view of a suture in accordance with the present disclosure attached to a needle.

### DETAILED DESCRIPTION

All composition percentages listed herein shall be understood to be by weight unless otherwise indicated. All quantities set forth below, except in the claims, shall be understood to be modified by the term “about”.

Antimicrobial characteristics may be imparted to a medical device in accordance with this disclosure by contacting the medical device with an antimicrobial solution containing at least one antimicrobial agent, at least one solvent, and at least one adherence-enhancing agent. The combination of the at least one antimicrobial agent and the at least one adherence-enhancing agent forms a complexed antimicrobial agent in the antimicrobial solution, which remains on at least a portion of a surface of the medical device after application of the antimicrobial solution and removal of the solvent. For those medical devices having both internal and external surfaces (e.g., stents, tubes, and/or multifilament sutures having an outer suture surface and internal surfaces found in the interstices between individual filaments making up the suture), the complexed antimicrobial agent may be found on a portion of the outer surface of such a device, the internal surfaces, or both.

The term “antimicrobial agent” as used herein includes antibiotics, antiseptics, disinfectants, and combinations thereof that are soluble in one or more solvents.

The term “adherence-enhancing agent” as used herein includes any material which increases the affinity of an antimicrobial agent for at least a portion of a surface of a medical device.

The term “complexed antimicrobial agent” as used herein includes the product of the combination of an antimicrobial agent and an adherence-enhancing agent. The “complexed antimicrobial agent” can include any form produced by the combination of the antimicrobial agent and adherence-enhancing agent, including salts, complexes, conjugates, micelles, etc.

Classes of antibiotics that can be used in the antimicrobial solution include tetracyclines like minocycline; rifamycins like rifampin; macrolides like erythromycin; penicillins like nafcillin; cephalosporins like cefazolin; beta-lactam antibiotics like imipenem and aztreonam; aminoglycosides like gentamicin and TOBRAMYCIN®; chloramphenicol; sulfonamides like sulfamethoxazole; glycopeptides like vancomycin; quinolones like ciprofloxacin; fusidic acid; trimethoprim; metronidazole; clindamycin; mupirocin; polyenes like amphotericin B; azoles like fluconazole; and beta-lactam inhibitors like sulbactam. Combinations of the foregoing may also be utilized in embodiments.

Examples of antiseptics and disinfectants which may be utilized in the antimicrobial solution include hexachlorophene; cationic biguanides like chlorhexidine and cyclohexidine; iodine and iodophores like povidone-iodine; halo-substituted phenolic compounds like PCMX (i.e., p-chloro-m-xylenol) and triclosan (i.e., 2,4,4′-trichloro-2′hydroxy-diphenylether); furan medical preparations like nitrofurantoin and nitrofurazone; methenamine; aldehydes like glutaraldehyde and formaldehyde; and alcohols. Combinations of the foregoing may also be utilized in embodiments. In embodiments, at least one of the antimicrobial agents is an antiseptic. In some embodiments, the antiseptic may be triclosan.

The antimicrobial solution generally contains from about 0.001 to about 25% of the antimicrobial agent by weight. The exact amount of the antimicrobial agent will depend on a number of factors, such as the particular agent used, the medical device being contacted, the choice of solvent employed, and the adherence-enhancing agent utilized.

As noted above, the antimicrobial solution also contains at least one adherence-enhancing agent which enhances the affinity of the antimicrobial agent for the medical device. Suitable adherence-enhancing agents include, but are not limited to, N-methylglucamine; L-arginine; sodium lauryl sulfate; cyclodextrins such as beta-cyclodextrin and hydroxypropyl-beta-cyclodextrin; ethanolamines such as ethanolamine, triethanolamine, and diethanolamine; and benzoates such as sodium benzoate and sodium methyl 4-hydroxybenzoate. Combinations of the foregoing adherence-enhancing agent(s) may be utilized in embodiments.

The adherence-enhancing agent may be present in amounts from about 0.01 percent to about 50 percent by weight of the antimicrobial solution, in embodiments from about 1 percent to about 25 percent by weight of the antimicrobial solution, and in embodiments from about 5 percent to about 15 percent by weight of the antimicrobial solution.

The antimicrobial solution can include any solvent or combination of solvents suitable for the chosen antimicrobial agent and adherence-enhancing agent. To be suitable, the solvent must (1) be miscible with the antimicrobial agent and adherence-enhancing agent, and (2) not appreciably affect the integrity of any material used to form the medical device to which the complexed antimicrobial agent is to be applied, such as a suture. In embodiments, the solvent may be a polar solvent. Some examples of suitable solvents include methylene chloride, chloroform, ethyl acetate, methyl acetate, N-methyl 2-pyrrolidone, 2-pyrrolidone, propylene glycol, tetrahydrofuran (THF), acetone, oleic acid, methyl ethyl ketone, water, and mixtures thereof.

The method of preparing the antimicrobial solution of the present disclosure is not critical and can be a relatively simple procedure. For example, the antimicrobial agent, solvent and adherence-enhancing agent may be combined with mixing at room temperature to produce the antimicrobial solution. In some embodiments, the solvent may be heated to enhance formation of the antimicrobial solution, provided that significant degradation of the antimicrobial activity of the antimicrobial agent is avoided.

Upon mixing in the antimicrobial solution, the adherence-enhancing agent combines with the antimicrobial agent to produce salts, micelles, complexes, and/or conjugates, which are sometimes referred to herein as a “complexed antimicrobial agent.” The complexed antimicrobial agent, by virtue of the adherence-enhancing agent, possesses greater affinity for the medical device to which it is applied, thereby reducing the loss of the antimicrobial agent from the surface of the medical device during handling, processing or storage, and thus providing for improved antimicrobial activity of the medical device upon implantation. The resulting medical device thus has improved shelf life without sacrificing its antimicrobial properties and lower amounts of antimicrobial agent may be utilized to achieve the desired antimicrobial effect upon implantation of the medical device in vivo.

Moreover, the adherence-enhancing agents utilized in accordance with the present disclosure can, in some embodiments, increase the solubility of the antimicrobial agent in the solvent utilized to form the antimicrobial solution. Without wishing to be bound by any theory, it is believed that the salts, micelles, complexes, and/or conjugates formed when the adherence-enhancing agent combines with the antimicrobial agent to form the complexed antimicrobial agent may enhance the solubility of the antimicrobial agent. By enhancing the solubility of the antimicrobial agent in the antimicrobial solution, lower amounts of antimicrobial agent may be needed to obtain the desired amount of antimicrobial agent upon the medical device, which reduces the amount of antimicrobial agent required to achieve the desired antimicrobial effect upon implantation of the medical device in vivo.

Any technique within the purview of those skilled in the art may be employed to apply the antimicrobial solution to the medical device. Suitable techniques include dipping, spraying, wiping and brushing. In embodiments, the antimicrobial solution may be applied to the medical device in its final form.

The amount of the antimicrobial solution applied to a medical device should be an effective amount to provide antimicrobial properties to the medical device. The exact amount will depend upon the configuration of the medical device and the formulation of the solution. In embodiments, for a suture, the antimicrobial solution may be applied in an amount from about 0.001 to about 25 weight percent by weight of the suture.

Since the antimicrobial solution contains a solvent, a curing step may be employed in embodiments to remove the solvent, leaving the complexed antimicrobial agent on the suture. Suitable curing steps for removal of the solvent include, but are not limited to, evaporation and/or lyophilization. Upon removal of the solvent, the complexed antimicrobial agent, i.e., the salt, conjugate, complex, micelle, etc., formed by the combination of the adherence-enhancing agent with the antimicrobial agent, remains bound to the medical device. In embodiments, the amount of the complexed antimicrobial agent on the medical device may be from about 0.01% by weight of the medical device to about 10% by weight of the medical device.

In embodiments, triclosan may be utilized as the antimicrobial agent. The desired amount of triclosan, which is slightly acidic, can be placed into a container, followed by the addition of the desired amount of solvent, such as methylene chloride, which has optionally been heated. A basic adherence-enhancing agent, such as an ethanolamine, may then be added. However, as one skilled in the art will appreciate, the order of addition of the ingredients is not important. The antimicrobial agent, adherence-enhancing agent and solvent may then be mixed thoroughly to combine the ingredients whereby the basic adherence-enhancing agent, for example, in embodiments an ethanolamine, forms a salt with the triclosan. The solution may be applied to a medical device, the solvent removed, and the resulting salt, i.e., the complexed antimicrobial agent, may be left on the medical device.

In another embodiment, a cyclodextrin may be utilized instead of an ethanolamine as the adherence-enhancing agent, in which case the cyclodextrin forms a micellular complex with the antimicrobial agent, e.g., triclosan, in the antimicrobial solution. The solution may be applied to a medical device, the solvent removed, and the resulting micellular complex, i.e., the complexed antimicrobial agent, may be left on the medical device.

Any medical device may be treated with a complexed antimicrobial agent in accordance with the present disclosure. Suitable medical devices include, for example, staples, clips, drug delivery devices, stents, pins, screws, and fibrous surgical articles such as sutures, prosthetic ligaments, prosthetic tendons, woven mesh, gauze, dressings, growth matrices and the like.

In one embodiment, the medical device treated in accordance the present disclosure may be a suture. Sutures in accordance with the present disclosure may be monofilament or multifilament and may be made of any conventional material, including both bioabsorbable and non-bioabsorbable materials, such as surgical gut, silk, cofton, polyolefins such as polypropylene, polyamides, polyglycolic acids, polyesters such as polyethylene terephthalate and glycolide-lactide copolymers, combinations thereof, etc.

In one embodiment, the suture may be made of a polyolefin. Suitable polyolefins include polyethylene, polypropylene, copolymers of polyethylene and polypropylene, and blends of polyethylene and polypropylene. In some embodiments, polypropylene can be utilized to form the suture. The polypropylene can be isotactic polypropylene or a mixture of isotactic and syndiotactic or atactic polypropylene.

In another embodiment, the suture may be made from synthetic absorbable polymers such as those made from glycolide, lactide, caprolactone, alkylene carbonates (i.e., trimethylene carbonate, tetramethylene carbonate, etc.), dioxanones, and copolymers and combinations thereof. In embodiments, a suture may include glycolide and lactide based polyesters, in embodiments copolymers of glycolide and lactide.

As noted above, the suture can be monofilament or multifilament. Where the suture is a monofilament, methods for producing such sutures are within the purview of those skilled in the art. Such methods include forming a suture material, such as a polyolefin resin, and extruding, drawing and annealing the resin to form the monofilament.

Where the sutures are made of multiple filaments, the suture can be made using any technique within the purview of those skilled in the art such as, for example, braiding, weaving or knitting. The filaments may also be combined to produce a non-woven suture. The filaments themselves may be drawn, oriented, crinkled, twisted, commingled or air entangled to form yarns as part of the suture forming process.

In embodiments a multifilament suture of the present disclosure can be produced by braiding. The braiding can be done by any method within the purview of those skilled in the art. For example, braid constructions for sutures and other medical devices are described in U.S. Pat. Nos. 5,019,093, 5,059,213, 5,133,738, 5,181,923, 5,226,912, 5,261,886, 5,306,289, 5,318,575, 5,370,031, 5,383,387, 5,662,682, 5,667,528, and 6,203,564, the entire disclosures of each of which are incorporated by reference herein. Once the suture is constructed, it can be sterilized by any means known to those skilled in the art.

In some cases a tubular braid, or sheath, can be constructed about a core structure which is fed through the center of a braider. Known tubular braided sutures, including those possessing cores, are disclosed, e.g., in U.S. Pat. Nos. 3,187,752, 3,565,077, 4,014,973, 4,043,344, and 4,047,533.

Medical devices of the present disclosure may also possess a coating to enhance their physical properties. Many suitable coatings are within the purview of those skilled in the art, as are methods for application of coatings to medical devices. In one embodiment, the coating may include a film-forming polymer. Film-forming polymers which may be utilized in the coating are within the purview of those skilled in the art and include glycolide, lactide, caprolactone, trimethylene carbonate, dioxanones, dioxepanones, etc., and copolymers and combinations thereof.

In embodiments, the film-forming polymer includes a caprolactone containing copolymer as described in U.S. Pat. No. 5,716,376, the entire disclosure of which is incorporated by reference herein. Such a caprolactone containing copolymer can be obtained by polymerizing a major amount of epsilon-caprolactone and a minor amount of at least one other copolymerizable monomer or mixture of such monomers in the presence of a polyhydric alcohol initiator.

Monomers which can be copolymerized with epsilon-caprolactone include alkylene carbonates such as trimethylene carbonate, tetramethylene carbonate, dimethyl trimethylene carbonate; dioxanones; dioxepanones; absorbable cyclic amides; absorbable cyclic ether-esters derived from crown ethers; hydroxyacids capable of esterification, including alpha hydroxy acids (such as glycolic acid and lactic acid) and beta hydroxyacids (such as beta hydroxybutyric acid and gamma hydroxyvaleric acid); polyalkyl ethers (such as polyethylene glycol) and combinations thereof. In embodiments, glycolide can be utilized as the comonomer in the film-forming polymer.

Suitable polyhydric alcohol initiators which may be utilized in preparing the film-forming polymer include glycerol, trimethylolpropane, 1,2,4-butanetriol, 1,2,6-hexanetriol, triethanolamine, triisopropanolamine, erythritol, threitol, pentaerythritol, ribitol, arabinitol, xylitol, N,N,N′,N′-tetrakis(2-hydroxyethyl)ethylenediamine, N,N,N′,N′-tetrakis(2-hydroxypropyl)ethylenediamine, dipentaerythritol, allitol, dulcitol, glucitol, altritol, iditol, sorbitol, mannitol, inositol, and the like; with mannitol being used in some embodiments.

The polyhydric alcohol initiator can be employed in small amounts, e.g., from about 0.01 to about 5, and in embodiments from about 0.1 to about 3, weight percent of the total monomer mixture.

Where utilized, the film-forming copolymer can contain from about 70 to about 98, in embodiments from about 80 to about 95, weight percent epsilon-caprolactone derived units, the balance of the copolymer being derived from the other copolymerizable monomer(s), such as glycolide.

In one embodiment, a coating for a medical device can include a film-forming polymer combined with a fatty acid salt. Such coatings are described in U.S. Pat. No. 4,201,216. In other embodiments a film-forming polymer may be combined with a salt of a fatty acid ester. Suitable salts of fatty acid esters include those of the formula: 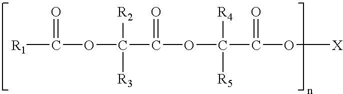
wherein x is an alkaline-earth metal or ion thereof, and R₁ is C₁₀ or greater alkyl, R₂ is H or C₁-C₃ alkyl, R₃ is H or C₁-C₃ alkyl, R₄ is H or C₁-C₃ alkyl, R₅ is H or C₁-C₃ alkyl, and n>1. Such suitable fatty acids include calcium, magnesium, aluminum, barium, or zinc stearoyl lactylate; calcium, magnesium, aluminum, barium, or zinc palmityl lactylate; calcium, magnesium, aluminum, barium, or zinc olelyl lactylate, and combinations thereof. In embodiments, a calcium stearoyl-2-lactylate (such as the calcium stearoyl-2-lactylate commercially available under the tradename VERV from American Ingredients Co., Kansas City, Mo.) may be utilized.

Where utilized, the film-forming polymer, such as the caprolactone/glycolide copolymer described above, can be present in an amount from about 45 to about 60 weight percent of the coating and the fatty acid salt or salt of a fatty acid ester can be present in an amount from about 40 to about 55 weight percent of the coating. In embodiments, the film-forming polymer, such as the caprolactone/glycolide copolymer described above, can be present in an amount from about 50 to about 55 weight percent of the coating and the fatty acid salt or salt of a fatty acid ester can be present in an amount from about 45 to about 50 weight percent of the coating.

Where a coating is used on the medical device, it should be understood that an antimicrobial solution in accordance with the present disclosure can be applied before, concurrently with, or after application of the coating. Thus, in some embodiments the antimicrobial solution may be applied to a coated medical device. In other embodiments, the anti-microbial solution may be mixed with the coating composition prior to application onto the medical device. In these embodiments, the coating and antimicrobial solution may be applied in a single step.

Contrary to other antimicrobial materials used with medical devices, the complexed antimicrobial agent of the present disclosure will not be lost due to evaporation, sublimation, volatilization, etc. during the subsequent handling, processing and storage of the subject medical device. However, upon application of the medical device in vivo, the adherence-enhancing agent portion of the complexed antimicrobial agent will hydrolyze, releasing the antimicrobial agent into the body.

The choice of adherence-enhancing agent utilized in the present disclosure may depend upon the selected antimicrobial agent and the medical device to which it may be applied. For example, where the antimicrobial agent is acidic in nature, an adherence-enhancing agent that is basic nature may be added in polar solvent to produce a salt. The resulting antimicrobial solution may then be applied to a medical device and the solvent removed, leaving the complexed antimicrobial agent, i.e., the salt produced by the combination of the adherence-enhancing agent and the antimicrobial agent, on the surface of the medical device. The resulting complexed antimicrobial agent is more hydrophilic than the antimicrobial agent alone, which results in greater affinity of the complexed antimicrobial agent for the medical device and/or any coating thereon, especially where the medical device is made of a polyester or possesses a synthetic film-forming coating as described above.

Similarly, in other embodiments a micellular complex could be formed between the antimicrobial agent and the adherence-enhancing agent. For example, where the antimicrobial solution includes an antimicrobial agent combined with a cyclic sugar derivative such as a cyclodextrin, a micellular complex may form between the antimicrobial agent and cyclodextrin which will remain on the surface of the medical device upon removal of the solvent. The complexed antimicrobial agent, in this case the micellular complex, will not migrate through the medical device and, similar to the salts described above, the hydrophilic portion of the complexed antimicrobial agent, i.e., the micelle, will have greater affinity for the medical device than the antimicrobial agent alone, especially where the medical device is made of a polyester or possesses a synthetic film-forming coating as described above.

Thus, while conventional antimicrobial agents may be undesirably lost from medical devices when applied by themselves, the complexed antimicrobial agents of the present disclosure remain attached to the surface of the medical device during the processing, handling, and storage of the device. This minimizes the loss of antimicrobial agent to the packaging of the medical device, the environment, etc. However, upon placement of the antimicrobial medical device in vivo, the complexed antimicrobial agent hydrolyzes, thereby releasing the antimicrobial agent from the surface of the medical device into the body.

In other embodiments, it may be desirable to include a pigment in the medical devices of the present disclosure. The term “pigment” herein is used interchangeably with the term “dye” and refers to such particles that absorb visible and/or infrared light. Suitable pigments are within the purview of those skilled in the art. Such pigments include, but are not limited to, carbon black, bone black, copper phthalocyanine dyes, D&C Green No. 6, D&C Violet No. 2, and combinations thereof as described in the handbook of U.S. Colorants for Food, Drugs and Cosmetics by Daniel M. Marrion (1979). Other dyes which may be used include indocyanine green, methylene blue, flourescein, india ink, Prussian blue, eosins, acridine, iron oxide, acramine yellow, and combinations thereof. Those skilled in the art will recognize that detectable moieties may also be utilized with such dyes. Such detectable moieties include, but are not limited to, fluorescers, bioluminescent and chemiluminescent molecules, combinations thereof, and the like.

Sutures in accordance with the present disclosure may be dyed by adding from about 0.1 percent to about 1.0 percent (by weight of the suture composition) dye, in embodiments from about 0.2 percent to about 0.6 percent dye.

As shown in the Figure, the suture disclosed herein, suture 101, may be attached to a surgical needle 100 by methods within the purview of those skilled in the art. As will be readily apparent to one skilled in the art, in some embodiments the needle itself may be similarly treated with an antimicrobial solution described above so that at least a portion of the needle surface possesses a complexed antimicrobial agent thereon.

Wounds may be sutured by approximating tissue and passing the needled suture through tissue to create wound closure. The needle is then typically removed from the suture and the suture tied.

Medical devices in accordance with this disclosure can be packaged and sterilized in accordance with techniques with the purview of those skilled in the art.

While the above description contains many specifics, these specifics should not be construed as limitations on the scope of the invention, but merely as exemplifications of particularly useful embodiments thereof. Those skilled in the art will envision many other possibilities within the scope and spirit of the invention as defined by the claims appended hereto.

What is claimed is:

1. An antimicrobial medical device comprising:
a medical device; and
a complexed antimicrobial agent located on at least a portion of a surface of the medical device.

2. An antimicrobial medical device as in claim 1 wherein the complexed antimicrobial agent comprises at least one adherence-enhancing agent selected from the group consisting of N-methylglucamine, L-arginine, sodium lauryl sulfate, beta-cyclodextrin, hydroxypropyl-beta-cyclodextrin, ethanolamine, triethanolamine, diethanolamine, sodium benzoate, sodium methyl 4-hydroxybenzoate, and combinations thereof.

3. An antimicrobial medical device as in claim 1 wherein the complexed antimicrobial agent comprises at least one antimicrobial agent selected from the group consisting of antibiotics, antiseptics, disinfectants and combinations thereof.

4. An antimicrobial medical device as in claim 3 wherein the at least one antimicrobial agent is an antiseptic selected from the group consisting of hexachlorophene, chlorhexidine, cyclohexidine, iodine, povidone-iodine, p-chloro-m-xylenol, triclosan, nitrofurantoin, nitrofurazone, methenamine, glutaraldehyde, formaldehyde, and alcohols.

5. An antimicrobial medical device as in claim 1 wherein the medical device is selected from the group consisting of staples, clips, drug delivery devices, stents, pins, screws, sutures, prosthetic ligaments, prosthetic tendons, woven mesh, gauze, dressings, and growth matrices.

6. An antimicrobial suture comprising:
at least one filament; and
a complexed antimicrobial agent located on at least a portion of a surface of the at least one filament.

7. An antimicrobial suture as in claim 6 wherein the suture is selected from the group consisting of monofilament sutures and multifilament sutures.

8. An antimicrobial suture as in claim 6 wherein the at least one filament comprises a polyolefin.

9. An antimicrobial suture as in claim 6 wherein the at least one filament is made from a synthetic absorbable polymer derived from one or more monomers selected from the group consisting of glycolide, lactide, caprolactone, trimethylene carbonate, tetramethylene carbonate, dioxanone, and combinations thereof.

10. An antimicrobial suture as in claim 6 wherein the at least one filament further comprises a coating comprising at least one film-forming polymer.

11. An antimicrobial suture as in claim 10 wherein the coating further comprises an additive selected from the group consisting of fatty acid salts and salts of fatty acid esters.

12. An antimicrobial suture as in claim 6 wherein the complexed antimicrobial agent comprises at least one antimicrobial agent selected from the group consisting of antibiotics, antiseptics, disinfectants and combinations thereof.

13. An antimicrobial suture as in claim 12 wherein the at least one antimicrobial agent is an antiseptic selected from the group consisting of hexachlorophene, chlorhexidine, cyclohexidine, iodine, povidone-iodine, p-chloro-m-xylenol, triclosan, nitrofurantoin, nitrofurazone, methenamine, glutaraldehyde, formaldehyde, and alcohols.

14. An antimicrobial suture as in claim 6 wherein the complexed antimicrobial agent comprises at least one adherence-enhancing agent selected from the group consisting of N-methylglucamine, L-arginine, sodium lauryl sulfate, beta-cyclodextrin, hydroxypropyl-beta-cyclodextrin, ethanolamine, triethanolamine, diethanolamine, sodium benzoate, sodium methyl 4-hydroxybenzoate, and combinations thereof.

15. A method comprising:
providing a suture having at least one filament;
applying an antimicrobial solution comprising at least one antimicrobial agent, at least one adherence-enhancing agent, and at least one solvent to the at least one filament; and
removing the at least one solvent leaving a complexed antimicrobial agent on at least a portion of a surface of the suture.

16. The method of claim 15 wherein the step of applying the antimicrobial solution utilizes an antimicrobial solution containing at least one antimicrobial agent selected from the group consisting of antibiotics, antiseptics, disinfectants and combinations thereof.

17. The method of claim 15 wherein the step of applying the antimicrobial solution utilizes an antimicrobial solution comprising triclosan as the at least one antimicrobial agent.

18. The method of claim 15 wherein the step of applying the antimicrobial solution utilizes an antimicrobial solution having at least one adherence-enhancing agent selected from the group consisting of N-methylglucamine, L-arginine, sodium lauryl sulfate, beta-cyclodextrin, hydroxypropyl-beta-cyclodextrin, ethanolamine, triethanolamine, diethanolamine, sodium benzoate, sodium methyl 4-hydroxybenzoate, and combinations thereof.

19. The method of claim 15 wherein the step of applying the antimicrobial solution utilizes an antimicrobial solution having an ethanolamine as the at least one adherence-enhancing agent.

20. The method of claim 15 wherein the step of applying the antimicrobial solution utilizes an antimicrobial solution having a cyclodextrin as the at least one adherence-enhancing agent.

21. The method of claim 15 wherein the step of applying the antimicrobial solution utilizes an antimicrobial solution having at least one solvent selected from the group consisting of methylene chloride, chloroform, ethyl acetate, methyl acetate, N-methyl 2-pyrrolidone, 2-pyrrolidone, propylene glycol, tetrahydrofuran (THF), acetone, oleic acid, methyl ethyl ketone, water, and mixtures thereof.

22. The method of claim 15 wherein the step of applying the antimicrobial solution comprises applying an antimicrobial solution containing triclosan as the at least one antimicrobial agent, an ethanolamine as the at least one adherence-enhancing agent, and methylene chloride as the at least one solvent.

23. The method of claim 15 wherein the step of applying the antimicrobial solution comprises applying an antimicrobial solution containing triclosan as the at least one antimicrobial agent, a cyclodextrin as the at least one adherence-enhancing agent, and methylene chloride as the at least one solvent.

24. A method of suturing a wound comprising:
a) providing a needled suture having on at least a portion of its surface a complexed antimicrobial agent and;
b) passing said needled suture through approximated wound tissue to create wound closure.
